(19) United States
(12) Patent Application Publication
(10) Pub. No.: US 2014/0186273 A1
(43) Pub. Date: Jul. 3, 2014

(54) Chitosan -Amelogenin Hydrogel for In Situ Enamel Growth

(71) Applicant: University of Southern California, Los Angeles, CA (US)

(72) Inventors: Janet MORADIAN-OLDAK, Los Angeles, CA (US); Qichao RUAN, Alhambra, CA (US)

(73) Assignee: University of Southern California, Los Angeles, CA (US)

(21) Appl. No.: 14/142,086

(22) Filed: Dec. 27, 2013

Related U.S. Application Data
(60) Provisional application No. 61/746,284, filed on Dec. 27, 2012.

(51) Int. Cl. A61K 8/73; A61K 8/66; A61Q 11/00; A61K 8/64

(52) U.S. Cl. CPC A61K 8/736; A61K 8/64; A61K 8/66; A61Q 11/00
USPC 424/50; 424/54

(57) ABSTRACT

A composition for re-constructing enamel-coated substrates such as a tooth which includes an amelogenin, chitosan, and water. The composition may also include compounds providing calcium and phosphate ions. Methods using the composition are also provided.

### CROSS-REFERENCE TO RELATED APPLICATIONS

This application claims the benefit of U.S. provisional application Ser. No. 61/746,284 filed Dec. 27, 2012, the disclosure of which is hereby incorporated in its entirety by reference herein.

### STATEMENT REGARDING FEDERALLY SPONSORED RESEARCH OR DEVELOPMENT

The invention was made with Government support under Contract No. DE-13414. The Government has certain rights to the invention.

### SEQUENCE LISTING

The text file usc0118-sequences_ST25, created Dec. 25, 2013, and of size 13 KB, filed herewith, is hereby incorporated by reference.

### TECHNICAL FIELD

In at least one aspect, the present invention is related to the methods of reconstructing enamel.

### BACKGROUND

Enamel is the exterior layer of the mammalian tooth and a hard biomaterial with significant resilience that protects the tooth from external physical and chemical damages. The remarkable mechanical properties of enamel are associated with its hierarchical structure from the nanoscale to the macroscale. The building blocks of enamel, the enamel rods, are densely packed arrays of elongated apatite crystals organized into an intricate interwoven structure. Cellular activities and protein-controlled process of mineralization are key to achieving such precisely organized structures. The proteins that mediate the mineralization of apatite crystals are gradually degraded and eventually removed during the enamel maturation. Mature enamel is non-living and cannot regenerate itself after substantial mineral loss, which often occurs as dental caries or erosion. Currently, the conventional treatments for carious lesions include refilling with amorphous materials like amalgam, ceramics or composite resin. However, even after those treatments, often secondary caries arise at the interface between the original enamel and filling materials due to weakened adhesion with time. There is, therefore, a need for alternative restorative material with improved adhesion to the tooth surface. One such alternative is a synthetic enamel-like material that can be prepared by biomimetic regrowth on the enamel surface.

Various biomimetic systems have been developed by investigators for repair of enamel defects, including liquids and pastes that contain nano-apatite or different organic additives for the remineralization of early, sub-micrometer-sized enamel lesions. A glycerine-enriched gelatin system was used to form dense fluorapatite layers on human enamel. Growth of enamel-like nanocrystals in small cavities from a paste containing fluoride-substituted hydroxyapatite was achieved in vitro. A compacted fluorapatite film with prism-like structure was synthesized on metal plates using a hydrothermal technique. Formation of enamel-like structures at ambient conditions was also performed in vitro using liquid and pastes with different organic additives. These investigations constitute significant progress in the study on enamel-like structures. Overall, the biomimetic strategies used still face an ongoing challenge in the fields of dentistry and material science.

Accordingly, there is a need for improved compositions for treating dental caries and related diseases.

### SUMMARY

The present invention solves one or more problems of the prior art by providing a composition for re-constructing enamel-coated substrates. In particular, the composition is useful for treating early dental carious and erosive lesions as well as enamel defects resulting from genetic diseases. The composition includes an amelogenin, chitosan, water, and a sufficient amount of a pH adjusting component if necessary such that the composition has a pH greater than about 6.0. Advantageously, the strategies set forth herein produce enamel-like materials that contain nano- and microstructures using amelogenin to control the crystallization of biomimetic calcium and phosphate. The results open up the promising possibility for the remodeling of complex enamel minerals in an amelogenin-containing system.

In another embodiment, a method using the composition set forth above is provided. The method includes a step of contacting an enamel coated substrate with a composition including an amelogenin, a chitosan, water, and a sufficient amount of a pH adjusting component if necessary such that the composition has a pH greater than about 6.0. The composition is allowed to air dry resulting in reconstruction of a portion of the enamel coated substrate.

In still another embodiment, a method for treating a subject is provided. The method includes a step of identifying a subject having dental caries, early dental carious and erosive lesions, or enamel defects resulting from genetic diseases. A tooth having dental caries, early dental carious and erosive lesions, or enamel defects resulting from genetic diseases is contacted with a composition including an amelogenin, a chitosan, water, a sufficient amount of a pH adjusting component if necessary such that the composition has a pH greater than about 6.0. The composition is allowed to air dry resulting in reconstruction of a portion of the tooth.

### BRIEF DESCRIPTION OF THE DRAWINGS

FIG. 1. a) Optical photograph of a tooth slice used in this work. b) SEM image of acid-etched enamel surface.

FIG. 2. SEM images of newly grown layer without amelogenin after remineralization in an artificial saliva solution for 7 days. a, c) top view, b, d) side view. a, b) Without, c, d) with chitosan gel.

FIG. 3. SEM and TEM images of natural enamel and newly-grown layer after remineralization in amelogenin-chitosan gel for 7 days. a) Microstructure of native enamel. Black arrows indicate the crystallographic orientations of the apatite crystallites in native enamel. b) After 7 days of remineralization with chitosan-amelogenin hydrogel, an enamel-like layer was formed on the surface of etched enamel; Inset shows thickness of newly-grown layer; Rectangle 1 and 2 represent the selected areas corresponding to b and c. White arrows indicate the apatite orientations in the newly-grown layer. c) The newly-grown layer was bound firmly to the surface of enamel. d) Bundles of organized crystals were found inside the repaired layer. The arrows present a typical bundle of paralleled crystals inside the newly-grown layer. Inset shows the homogeneous surface of the repaired layer. e) HRTEM image of a rod-like crystal taken from the area outlined by the red rectangle on the crystal bundle in the inset. The arrow indicates the crystallographic direction of an apatite crystal along the c-axes. HRTEM image represents a typical bundle of paralleled crystals. f) Selected area electron diffraction (SAED) image of the newly-grown layer. Inset shows TEM image of the repaired layer prepared by focused ion beam (FIB) milling.

FIG. 4. a) XRD spectra of newly-grown layer after remineralization in a chitosan gel a) with and b) without amelogenin for 7 days. b) EDS spectrum of repaired layer after remineralization in a chitosan gel with amelogenin for 7 days.

FIG. 5. CD and fluorescence spectra were measured with different mass ratio at pH a1-a2) 3.5, b1-b2) 5.5 and c) 8.0, revealing that the interaction between chitosan and amelogenin is pH dependent.

FIG. 6. a) TEM image of the original CS-AMEL hydrogel showing the elongated nanochain-like structure (white arrows). b) Cross-section SEM image of repaired layer after remineralization in amelogenin-chitosan gel for 3 days fused to the surface of the natural enamel. The white and black arrows indicate the crystallographic orientations of the crystals in newly-grown layer and natural enamel, respectively. The dot line shows the boundary of the natural enamel and the newly grown layer. c) HRTEM image taken from the interface between the enamel and regrown crystal, showing seamless growth of repaired crystal on the enamel. The black arrows indicate the interface between regrown and enamel crystals. The inset shows the fast Fourier transform (FFT) images corresponding to enamel and regrown crystals. d) Schematic illustration of the enamel repair process.

FIG. 7. SEM images of reconstructed enamel-like layers after ultrasonic treatment. a) backscattered electron image of the cross section, and b) second electron image of the surface of an ultrasonic-treated newly-grown layer obtained in a chitosan-amelogenin hydrogel. Inset shows the typical morphology of the surface at a higher magnification. c) backscattered electron image of the cross section of an ultrasonic-treated newly-grown layer obtained in a chitosan hydrogel without amelogenin.

FIG. 8. a) Hardness and elastic modulus of healthy enamel, etched enamel, and reconstructed enamel repaired by chitosan hydrogel with and without amelogenin. * p≦0.05 when compared with artificial caries; *** p<0.001 when compared with artificial caries. b) OD values of the overnight cultures of saliva bacteria in different LB media. ### p<0.001 when compared with LB control. The inset image shows the turbidity of LB medium with and without chitosan hydrogel.

FIG. 9. a-c SEM images of the newly grown layer after remineralization in CS-AMEL hydrogel with 1% m/v (a), 2% m/v (b) and 3% m/v (c) chitosan. Insets show the crystal morphology at high magnification. d XRD patterns of the newly grown layer after remineralization in CS-AMEL hydrogel with different concentrations of chitosan.

FIG. 10. a-c SEM images of the newly grown layer after remineralization in CS-AMEL hydrogel with 5.0 mM (a), 7.5 mM (b) and 10 mM (c) of Ca²⁺. Insets show the crystal morphology at high magnification. d XRD patterns of the newly grown layer remineralized in CS-AMEL hydrogel with different concentrations of Ca²⁺.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

The term “hydrogel” refers to a gel in which the dispersion medium is water.

The term “subject” refers to a human or animal, including all mammals such as primates (particularly higher primates) having dental caries, early dental carious and erosive lesions as well as enamel defects resulting from genetic diseases.

The term “amelogenin” refers to the closely related polypeptides involved in the formation of enamel and isoforms thereof. Amelogenin is exemplified by SEQ ID NOs: 1-8 (i.e., SEQ ID NO: 1, SEQ ID NO: 2, SEQ ID NO: 3, SEQ ID NO: 4, SEQ ID NO: 5, SEQ ID NO: 6, SEQ ID NO: 7, and/or SEQ ID NO: 8). In a refinement, this definition includes alternatively spliced isoforms such as LRAP (leucine rich amelogenin polypeptide). It should be appreciated that this definition includes polypeptides having 1 to 10 conservative substitutions of SEQ ID NOs: 1-8. In a refinement, this definition includes polypeptides having 1 to 3 conservative substitutions of SEQ ID NOs: 1-8. In another refinement, this definition includes polypeptide sequences having greater than 80 percent sequence identity to any of SEQ ID NOs: 1-8. In another refinement, this definition includes polypeptide sequences having greater than 95 percent sequence identity to any of SEQ ID NOs: 1-8.

In at least one aspect, a first composition for re-constructing enamel is provided. In particular, the composition is used for treating early dental carious and erosive lesions as well as enamel defects resulting from genetic diseases. The first composition includes an amelogenin, chitosan, and water. In a refinement, the composition includes a sufficient amount of a pH adjusting component such that the composition has a pH greater than about 6.0. Advantageously, the composition forms an organized mineral layer when contacting an enamel surface.

In a variation, the chitosan has a formula describe by a partially acetylated polysaccharide having formula I:

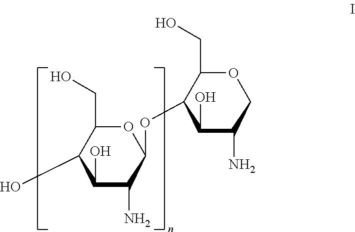

wherein n is from about 500 to 2500. In a refinement, the partially acetylated polysaccharide having formula I has a degree of acetylation less than about 35 percent. In a further refinement, the partially acetylated polysaccharide having formula I has a degree of acetylation from about 5 to 35 percent. The chitosan in general has a viscosity average molecular weight (Mv) from about 150,000 to 400,000 Daltons. In a refinement, the chitosan has an Mv from about 190,000 to 310,000 Daltons.

Examples of suitable amelogenins include, but are not limited to, porcine amelogenin rp172, mouse amelogenins, human amelogenins, etc., recombinant variations thereof, and isoforms thereof, and combinations thereof. Such isoforms include truncated amelogenin rP147 or an alternatively spliced isoform such as LRAP (leucine rich amelogenin polypeptide) having 59 amino acids. SEQ ID NOs: 1-8 provides specific examples of amelogenins.

In a variation of the present embodiment, the polypeptides with SEQ ID NOs: 1-8 have an amino acid sequence that is at least 80 percent identical to the amino acid sequence set forth as the polypeptides with SEQ ID NOs: 1-8. In other refinements, the polypeptides with SEQ ID NOs: 1-8 have an amino acid sequence that is at least, in order of increasing preference, 85%, 90%. 91%, 92%, 93%, 94%, 95%, 96%, 97%, 98%, 98.5% or 99% percent identical to the amino acid sequence set forth as SEQ ID NOs: 1-8. Thus, the present invention also encompasses the use of sequences having a degree of sequence identity with the SEQ ID NOs: 1-8. Herein, the term “sequence identity” means a polypeptide having a certain similarity with the subject amino acid sequence. The similar amino acid sequence should provide and/or encode a polypeptide which retains the functional activity of the sequence. A similar sequence includes an amino acid sequence is at least, in order of increasing preference, 80%, 85%, 90%. 91%, 92%, 93%, 94%, 95%, 96%, 97%, 98%, 98.5% or 99%, identical to the subject polypeptide sequence.

In a refinement, sequence identity comparisons are conducted as is well known in the art using sequence comparison computer programs that use algorithms to align two or more sequences using a scoring system that rewards alignment of identical or similar amino acids and penalizes substitutions of non-similar amino acids and gaps. Computer programs for carrying out alignments include, but are not limited to, BLASTP which is publicly available from the National Center for Biotechnology Information (www.ncbi.nlm.nih.gov/). These programs calculate percent sequence identity and report the determined value. It is preferred to use the default values when using such software for amino acid sequence alignments. BLASTP is preferred to determine amino acid sequence identity between the subject polypeptide sequences SEQ ID NOs: 1-8 and a candidate polypeptide sequence according to the present invention. Details of the BLASTP algorithm are set forth in D. W. Mount “Bioinformatics: Sequence and Genome Analysis,” Cold Spring Harbor Press (2004). A particularly preferred set of parameters for the BlastP alignment includes a Blossum 62 scoring matrix with a gap penalty of 11, a gap extend penalty of 1, and conditional adjustments set to conditional compositional score matrix adjustment. Other preferred parameters for the BlastP alignment are an expected threshold of 10 and a word size of 3.

In another variation, the amelogenins, in particular the polypeptides, described by SEQ ID NOs: 1-8, include deletions, insertions or substitutions of amino acid residues which result in a functionally equivalent protein. Preferably, the substitutions are conservative being similar with respect to polarity, charge, solubility, hydrophobicity, hydrophiliticity, and/or the amphipathic nature of the residues, while preserving the functionality of forming enamel. Conservative substitutions that may be made are, for example, substitutions of aliphatic amino acids (Alanine, Valine, Leucine, Isoleucine), polar amino acids (Glutamine, Asparagine, Serine, Threonine), acidic amino acids (glutamic acid and aspartic acid), basic amino acids (Arginine, Lysine and Histidine), aromatic amino acids (Phenylalanine, Tryptophan and Tyrosine), large amino acids (Phenylalanine and Tryptophan), small amino acids (Glycine, Alanine) and hydroxyl amino acids (Serine, Threonine). In a refinement, an amelogenin includes polypeptides having 1 to 10 conservative substitutions of SEQ ID NOs: 1-8. In a refinement, this definition includes polypeptides having 1 to 3 conservative substitutions of SEQ ID NOs: 1-8.

Typically, the amelogenin is present in an amount from about 0.03 percent to about 0.4 percent of the total weight of the first composition and the chitosan is present in an amount from about 0.2 to about 3 percent of the total weight of the first composition with the balance being water.

In a variation, the first composition further includes a phosphate-containing compound that provides phosphate ions when dissolved in water. Examples of such phosphate-containing compound include, but are not limited to, Na₂HPO₄ which provides hydrogen phosphate ions (HPO₄²⁻). Typically, the amount of the phosphate-containing compound is present in an amount from about 0.01 percent to about 0.2 percent of the total weight of the first composition. The pH of the first composition is set to a pH equal to or greater than 6.0. A base such as NaOH may be used to adjust the pH. The first composition is advantageously a hydrogel that is useful for re-construction of enamel while inhibiting bacterial growth.

The first composition is advantageously used to re-construct enamel by contacting an enamel coated substrate (e.g., a tooth) with the composition and then allowing the composition to air dry. The enamel coated substrate is optionally contacted with a base (NaOH) for a first period of time (0.5 to 2 hours) to adjust the pH to a value of 6.0 or greater. The enamel coated substrate is then contacted with a calcium-containing compound (e.g., CaCl₂) that provides calcium ion when dissolved in water for a second period of time (e.g., 5 minutes to an hour). The enamel coated substrate is then optionally rinsed and then contacted with saliva for an extended period of time (>24 hours).

In another aspect, a second composition for re-constructing enamel is provided. The second composition includes an amelogenin, chitosan, a calcium-containing compound that provides calcium ion when dissolved in water (e.g., CaCl₂), a phosphate-containing compound (e.g., Na₂HPO₄) that provides phosphate ions and/or hydrogen phosphate ions when dissolved in water, and water. Examples of suitable amelogenins are set forth above. Typically, the amelogenin is present in an amount from about 0.03 percent to about 0.2 percent of the total weight of the second composition and the chitosan is present in an amount from about 0.2 to about 3 percent of the total weight of the second composition, and the calcium containing compound is present in an amount from about 0.01 percent to about 0.2 percent of the total weight of the second composition with the balance being water. The details regarding the chitosan are also set forth above. In a variation, the second composition further includes a compound such as Na₂HPO₄ providing hydrogen phosphate ions (HPO₄²⁻). Typically, the amount of the compound providing phosphate ions is present in an amount from about 0.01 percent to about 0.2 percent of the total weight of the second composition. The pH of the first composition is set to a pH equal to or greater than 6.0. A base such as NaOH may be used to adjust the pH to a value of 6.0 or greater. The second composition is advantageously a hydrogel that is useful for re-construction of enamel while inhibiting bacterial growth.

The second composition is advantageously used to re-construct enamel by contacting an enamel coated substrate (e.g., a tooth) with the composition and then allowing the composition to air dry. The enamel coated substrate is optionally contacted with a base (NaOH) for a first period of time (0.5 to 2 hours) to adjust the pH to a value of 6.0 or greater. The enamel coated substrate is then optionally rinsed and then contacted with saliva for an extended period of time (>24 hours).

In one or more variations of the first composition and the second composition, additional protein components are included. Examples of such additional components include, but are not limited to, enamelin, ameloblastin, enamel proteases, chitosanolytic enzymes, and combinations thereof. Suitable enamel proteases include, but are not limited to, Kallikrein-related peptidase 4 (KLK-4) and matrix metalloproteinase-20 (MMP-20). In a refinement, these additional components are individually or collectively present in an amount from about 0.005 percent to about 0.1 percent of the total weight of the first composition

In another aspect, a re-constructed enamel layer is provided. The re-constructed layer is formed from the composition and methods set forth above. In a refinement, the re-constructed enamel includes bundles of fluoridated calcium hydroxyapatite crystals that are organized with their crystallographic c-axes parallel to each other, are fused with existing enamel crystals forming a dense interface, and have elongated prism-like shapes.

As set forth above, the compositions of the invention are advantageously used to re-construct an enamel coated substrate. The method includes a step of contacting an enamel coated substrate with a composition including an amelogenin, a chitosan, water, a sufficient amount of a pH adjusting component if necessary such that the composition has a pH greater than about 6.0. The composition is allowed to air dry resulting in reconstruction of a portion of the enamel coated substrate. In a variation, the composition includes a calcium-containing compound that provides calcium ion when dissolved in water and a phosphate containing compound that provides phosphate ions when dissolved in water as set forth above. In another variation, the method further includes contacting the substrate with a base for a first period of time. In other variations, the method includes a step of contacting the substrate with a component selected from the group consisting of calcium-containing compound, a phosphate containing compound, and combinations thereof for a second period of time. The enamel coated substrate is usually contacted with saliva or a saliva mimicking solution. Such solutions include water and a component selected from the group consisting of electrolytes, mucus, glycoproteins, enzymes, antibacterial compounds, and combinations thereof. It is readily appreciated that the methods are advantageously used to treat a subject having dental caries, early dental carious and erosive lesions, and enamel defects resulting from genetic diseases.

Examples of applying the compositions set forth above are as follows:

### Method I: Diffusion Method.

1. 0.5 ml amelogenin-containing chitosan hydrogel were prepared by mixing of 475 ul chitosan solution (1% m/v), 25 ul Na2HPO4 solution (1 M), and 200 ug amelogenin followed by stirring at room-temperature overnight.
2. 20 ul chitosan-based hydrogel was spread on the acid-etched tooth slice, and dried in the air.
3. The tooth slice was soaked in the 1 M NaOH solution at room temperature for 1 h.
4. The tooth slice was rinsed with the distilled water, and then soaked in 0.1 M CaCl2.2H2O solution for 30 min.
5. After rinsed in distilled water, the tooth slice was soaked in 30 ml of artificial saliva solution at 37° C. for several days.

### Method II: Mixing Method.

1. 1 ml amelogenin-containing chitosan hydrogel were prepared by mixing of 960 ul chitosan solution (1% m/v), 15 ul Na2HPO4 solution (0.1 M), 25 ul CaCl2 (0.1 M) and 200 ug amelogenin.
2. The pH of chitosan-based hydrogel was adjusted to 6.5 by 1 M NaOH solution.
3. 20 ul chitosan-based hydrogel was spread on the acid-etched tooth slice, and dried in the air.
4. The tooth slice was soaked in 30 ml of artificial saliva solution at 37° C. for several days.
(note, method II is used in the experimental section set forth below).

The composition and methods of the invention are further illustrated in the following examples. These are provided by way of illustration and are not intended in any way to limit the scope of the invention.

Experimental showing Amelogenin-chitosan matrix promoting assembly of an enamel-like layer with a dense interface

In natural enamel, the formation of apatite crystals occurs in an amelogenin-rich matrix that plays a critical role in controlling the oriented and elongated growth of apatite crystals. Accordingly, we have used several strategies to prepare enamel-like materials that contain nano- and microstructures using amelogenin to control the crystallization of biomimetic calcium and phosphate. The results have opened up the promising possibility of remodeling of complex enamel minerals in an amelogenin-containing system.

Here we report development of a new amelogenin-containing chitosan (CS-AMEL) hydrogel to synthesize an organized, enamel-like mineralized layer on an acid-etched enamel surface used as an early caries model. Compared to the previous amelogenin-containing system that was developed, CS-AMEL is easier to handle in the clinic. It is biocompatible, biodegradable, and has unique antimicrobial and adhesion properties that are practical for dental applications. Chitosan has been observed to have antimicrobial activity against fungi, viruses, and some bacteria, including streptococci and lactobacilli, which are known as the principal etiological factors of dental caries. Therefore, we expect that the “synthetic enamel” formed in CS-AMEL hydrogel will have antimicrobial properties that can prevent bacterial infection and subsequent demineralization. In addition, chitosan is mucoadhesive to both hard and soft surfaces. Importantly, the newly formed crystals in CS-AMEL hydrogel grow directly on the original enamel, achieving a complete adhesion of the repaired layer to the natural enamel with a dense interface. The robust attachment of the newly grown layer demonstrated in the present work can potentially improve the durability of restorations and avoid the formation of new caries at the margin of the restoration.

### 2. Materials and Methods

### 2.1. Amelogenin Preparation

Recombinant full-length porcine amelogenin rP172 was expressed in E.coli and purified as described previously. The rP172 protein has 172 amino acids and is an analogue of the full-length native porcine P173, but lacking the N-terminal methionine as well as a phosphate group on Ser16.

### 2.2. Tooth Slice Preparation

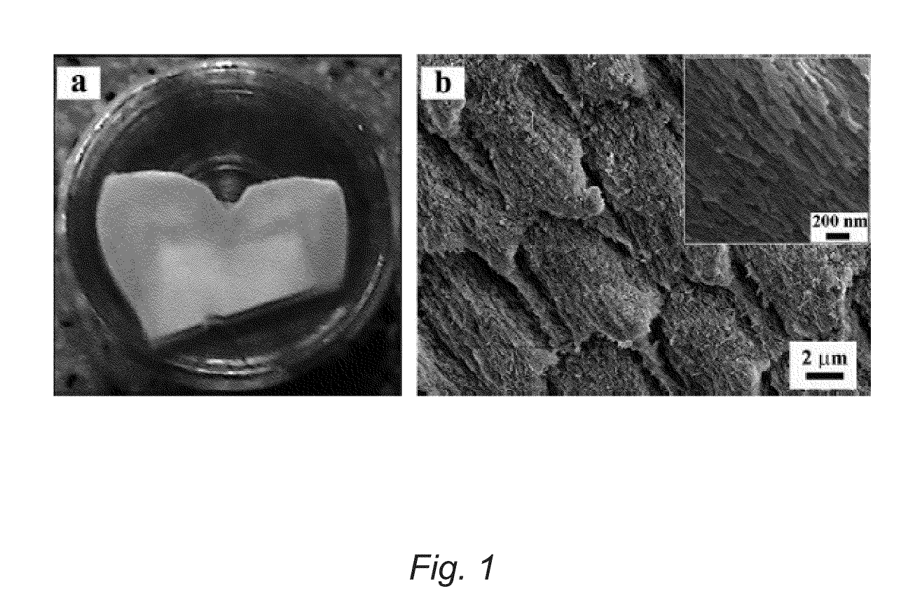

Human third molars (extracted following the standard procedures for extraction at the Ostrow School of Dentistry of the University of Southern California and handled with approval of the Institutional Review Board) without any restored caries were selected. Slices 0.1-0.2 cm in thickness (FIG. 1a), were cut longitudinally using a low speed diamond saw cooled by water. To simulate early caries lesions, tooth slices were acid-etched with 30% phosphoric acid for 30 s and rinsed with deionized water.

### 2.3. Etched Enamel Repaired by Amelogenin-Containing Chitosan Hydrogel

Amelogenin-containing chitosan hydrogel was prepared by mixing chitosan (medium molecular weight, 75-85% deacetylated, Sigma-Aldrich) solution (960 μl, 1% m/v), Na₂HPO₄ (15 μl, 0.1 M), CaCl₂ (25 μl, 0.1 M) and amelogenin rP172 (200 μg), followed by stirring at room temperature overnight, and the pH value was adjusted to 6.5 with 1M NaOH. Twenty μl of chitosan-based hydrogel was carefully applied onto the enamel surface and dried in air at room temperature. The tooth slices were then immersed in 30 ml of artificial saliva solution (MgCl₂ 0.2 mM, CaCl₂.H₂O 1 mM, HEPES buffer 20 mM, KH₂PO₄ 4 mM, KCl 16 mM, NH₄Cl 4.5 mM, NaF 300 ppm, pH=7.0, adjusted with 1 M NaOH) at 37° C. for 7 days. After the allotted time, the tooth slice was removed from the solution, rinsed with running deionized water for 50 s and air-dried.

### 2.4. Characterization

SEM imaging was performed on a field emission scanning electron microscope (JEOL JSM-7001F), operating at an accelerating voltage of 10 kV. X-ray diffraction (XRD) patterns were recorded on a Rigaku Diffractometer with Cu Kr radiation (λ=1.542 Å) operating at 70 kV and 50 mA with a step size of 0.02°, at a scanning rate of 0.1° s⁻¹ in a 2θrange from 10° to 60°. HRTEM images were obtained on a JEOL JEM-2100 microscope using an accelerating voltage of 200 kV. The hardness and elastic modulus were measured at 20 test points in each sample (n=3) using a Nano-indenter (Agilent-MTS XP) with a Berkovich tip. Circular dichroism spectropolarimetry (CD) was performed using a JASCO J-815 spectropolarimeter (JASCO, Easton, Md., USA). The spectra were recorded between 190 and 260 nm with a step size of 0.5 nm and a scan rate of 50 nm/min. Fluorescence spectroscopy was performed using a PTI QuantaMaster QM-4SE spectrofluorometer (PTI, Birmingham, N.J., USA). The amelogenin solutions were excited at 290 nm. The emission spectra were monitored between 300 and 400 nm with a step size of 1 nm.

### 2.5. Antimicrobial Evaluation

Human saliva was collected as described in the literature for the antimicrobial experimentation: Healthy adults were chosen as subjects for collecting saliva. Subjects were asked to refrain from eating, drinking, and oral hygiene procedures for at least 1 h prior to the collection. Subjects were given distilled drinking water and asked to rinse their mouths out with it for 1 min. Five minutes after this oral rinse, subjects were asked to spit into a 50 ml sterile tube, which was placed on ice while collecting more saliva. Subjects were instructed to tilt the head forward and let the saliva run naturally to the front of the mouth; Upon the collection of approximately 5 ml volume of saliva from the subject, the saliva sample was taken to the laboratory immediately for processing. 20 μl of saliva were added to tubes with 1 ml of lysogeny broth (LB) media containing chitosan-amelogenin hydrogel or amelogenin, and then incubated at 37° C. overnight. The OD₆₀₀ of the overnight cultures was measured by a Beckman DU-640 Spectrophotometer.

### 2.6 Statistical Analysis

Enamel remineralization experiments were repeated three times. The mechanical tests and antimicrobial experiments were conducted in triplicate and data were expressed as mean±standard deviations. For mechanical testing the Student's t-test was applied to identify differences in the hardness and elastic modulus between etched and repaired enamel of samples (n=3). For the antimicrobial experiments (n=3) the OD values were compared between control and samples containing chitosan-amelogenin hydrogel or amelogenin by the same test. In all experiments the differences were considered statistically significant at p≦0.05 and highly significant at p<0.001. All the statistical analyses were carried out using Origin 8.0 (Origin lab, Northampton, Mass.) and Microsoft Office Excel 2007.

### 3. Results and Discussion

3.1. Enamel Remineralization without CS-AMEL Hydrogel

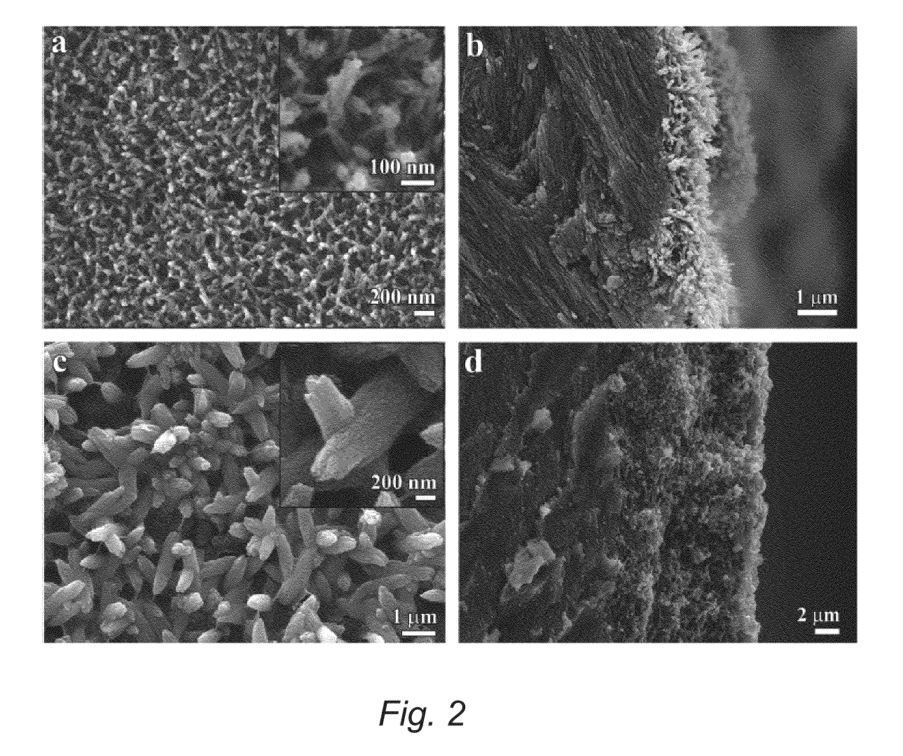

Dental caries is caused by an imbalance in the dynamic process of demineralization-remineralization of enamel. Enamel demineralization occurs at a low pH caused by acids of bacterial origin. To produce artificial caries, a tooth slice was etched with 30% phosphoric acid. When examined, the etched enamel crystals were seen to be discontinuous and broken, resembling crystals from carious enamel (FIG. 1b). Although the calcium and phosphate ions in the saliva permit the recovery of some lost enamel mineral, the remarkably organized structure of enamel cannot be regained without protein mediation. We prepared an artificial saliva (AS) solution to simulate the oral environment for enamel remineralization. After soaking in AS solution alone for 7 days, a calcium phosphate coating with a thickness of 1 μm was formed on the surface of enamel. As shown in FIG. 2a and b, the remineralized crystals had rod-like structure and the coating was porous. A similar layer but with a thickness of 10 μm was formed on the etched enamel soaked in chitosan hydrogel without amelogenin. This remineralized apatite layer also consisted of loosely packed crystals with a porous structure (FIGS. 2c and d). These porous layers did not resemble natural enamel structure, which has a high packing density of apatite crystals.

3.2. Enamel Remineralization with CS-AMEL Hydrogel

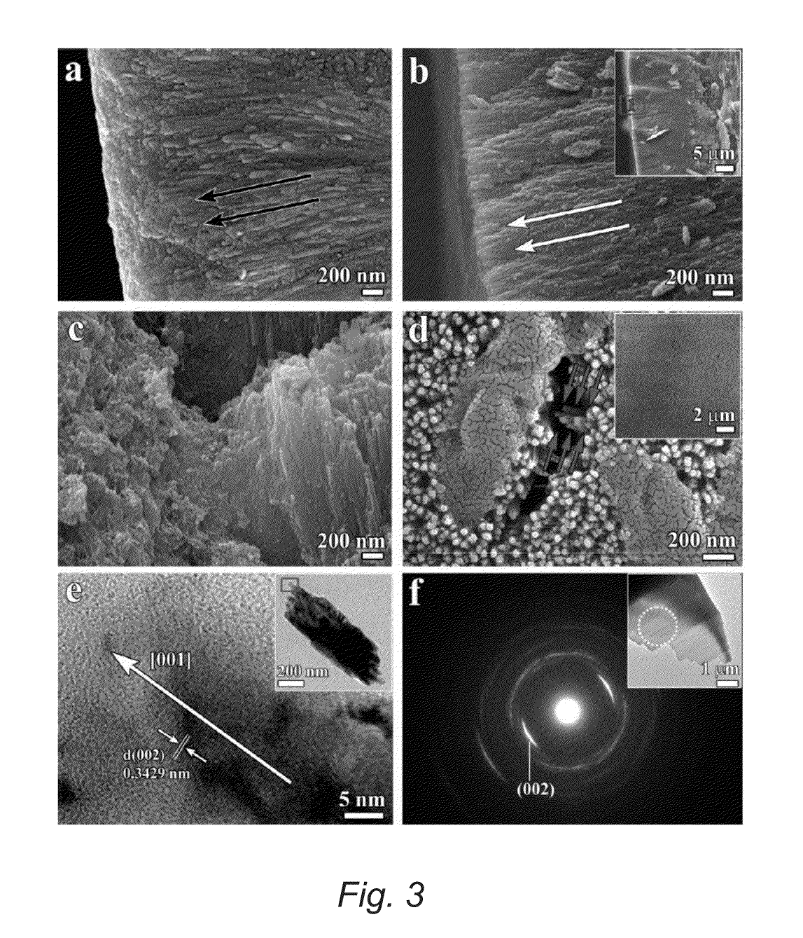

FIG. 3 shows the microstructures of human molar enamel and the newly-grown layer on an etched enamel surface soaked for 7 days in CS-AMEL hydrogel. At the nanoscale (FIG. 3a), natural enamel is made of highly organized arrays of apatite crystallites growing preferentially along the c-axis, perpendicular to the surface (black arrows in FIG. 3a). After mineralization for 7 days, similar organized crystals were formed on the etched enamel surface treated by CS-AMEL hydrogel. The crystals grown in CS-AMEL hydrogel were composed of numerous nanorods oriented preferentially along the c-axis with a diameter of ˜50 nm, nearly parallel to each other in the longitudinal direction (white arrows in FIG. 3b). The newly-grown layer, with a thickness of 15 μm, was tightly bound to the surface of the natural enamel (FIG. 3b inset). Examination at higher magnification revealed no obvious boundary at the interface (FIG. 3c). The bulk of the newly-grown layer contained needle-like crystals that were bundled to form a fundamental organization unit analogous to that of natural enamel crystallites (FIGS. 3d and 3e). The high-resolution transmission electron microscopy (HRTEM) image showed clear lattice fringes perpendicular to the nanorod axis with an interplanar spacing of d=0.3429 nm, in accordance with the distance between the (002) crystal planes of hydroxyapatite (JCPDS 09-0432), which suggests that the nanorods formed in CS-AMEL hydrogel grow in the [001] direction (white arrow in FIG. 3e). Selected area electron diffraction (SAED) of the newly-grown layer resulted in an arc-shaped pattern along the (002) diffraction plane, indicating a hierarchical alignment of the c-axes of the newly formed crystals (FIG. 3f).

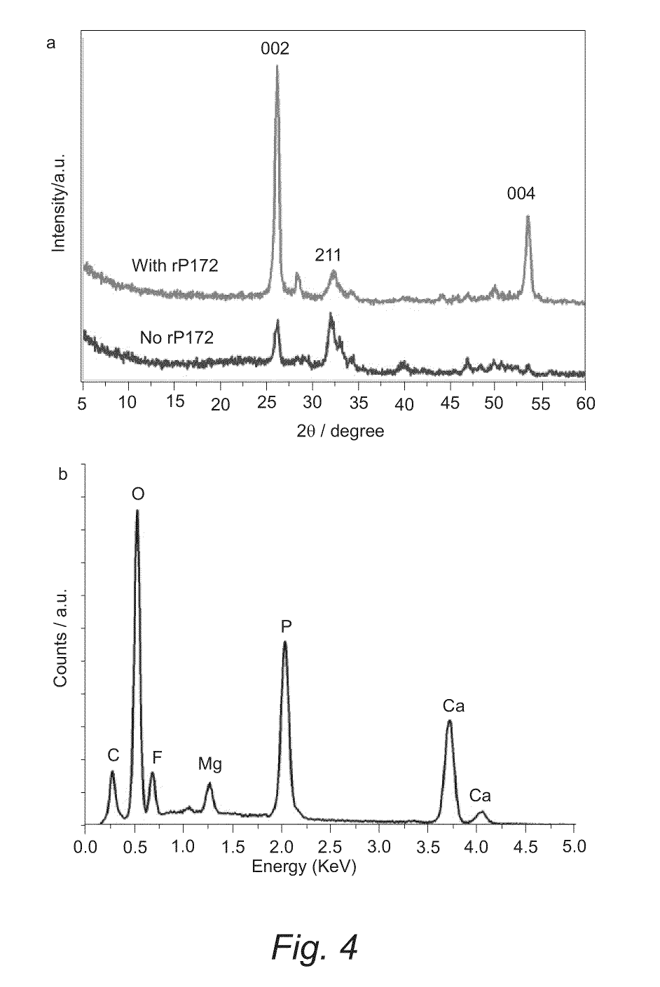

The orientation and composition of the newly-grown crystals were further confirmed by x-ray diffraction (XRD) and energy dispersion spectroscopy (EDS) (FIG. 4). All of the diffraction peaks can be readily indexed to hexagonal phase hydroxyapatite (JCPDS 09-0432) crystals. The unsplit diffraction peak around 2θ=32° indicates the poor crystallinity of newly-formed apatite in CS-AMEL hydrogel (FIG. 4a). Sharp and intense 002 and 004 peaks indicates that the (001) faces are parallel to the surface (FIG. 4a), i.e., the crystals align in an orderly fashion along the crystallographic c-axis, in accordance with the microstructure obtained by SEM and TEM observations (FIG. 3). EDS revealed the presence of calcium, phosphate and fluorine ions in the newly-grown layer (FIG. 4b). The structural and compositional analyses indicated that the newly-formed layer contain fluoridated hydroxyapatite (FAP) with poor crystallinity.

3.3. Functions of Chitosan and Amelogenin in Enamel Remineralization with CS-AMEL Hydrogel

Comparing the morphologies of remineralized layers formed in chitosan hydrogel with and without amelogenin, we observed that disordered structures with porous morphology were formed without the protein (FIG. 2), but ordered enamel-like structures were obtained in the presence of amelogenin (FIGS. 3b-f). These results indicate that amelogenin mediation is an essential factor for the formation of the orderly enamel-like structure in the chitosan hydrogel system. Although chitosan hydrogel has also been reported as a mineralization matrix because of its charged surface, the chitosan molecules were not found to affect the function of amelogenin during the synthesis of the repaired layer.

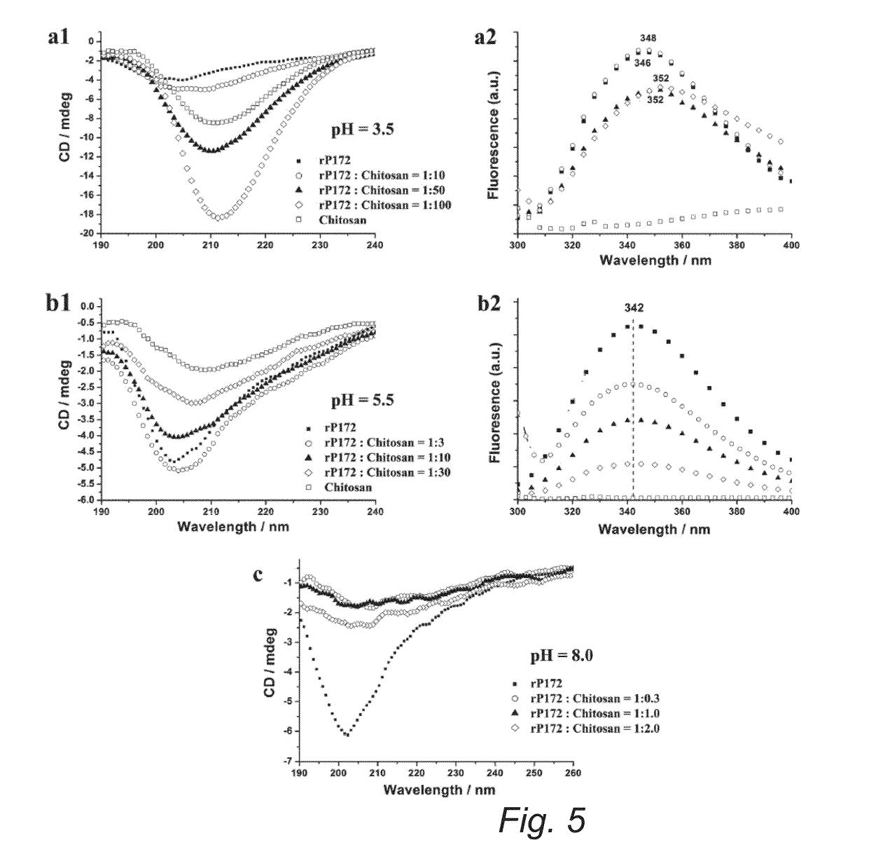

The chitosan-amelogenin interaction was studied by using Circular Dichroism (CD) and Fluorescence spectroscopy at pH 3.5, 5.5 and 8.0 (FIG. 5). All the CD spectra of pure amelogenin showed negative ellipticities around 203 nm, which were characteristic of unordered polyproline type II structures. At pH 3.5, the intensity of the minima gradually increased and the trough shifted to a higher wavelength with increasing ratios of chitosan to amelogenin, indicating a possible change in the conformation of amelogenin in the presence of chitosan. In the corresponding fluorescence spectra, red shifts of the emission maxima were also observed with increasing amounts of chitosan, indicating the exposure of tryptophan residues belonging to the amelogenin (FIG. 5a). These changes in the CD and fluorescence spectra clearly illustrated that there was a direct interaction between amelogenin and chitosan at pH 3.5. At pH 5.5, both the intensity of the negative dichroic signals and the positions of their minima were changed with the addition of chitosan to the amelogenin; however, there was no shift in the fluorescence spectra (FIG. 5b). When the pH reached 8.0, it was difficult to find the dichroic signal or the emission band in CD and fluorescence spectra of amelogenin in association with chitosan (FIG. 5c). The results from the CD and fluorescence spectra revealed that the interaction between chitosan and amelogenin is dependent on pH. At pH below the pKa of chitosan (6.5), the amino groups were almost completely ionized, and the charge density of chitosan increased; thus chitosan interacted with amelogenin through electrostatic interaction. In contrast, when pH was higher than 5.5, chitosan's interaction was weak because of its low solubility and deprotonation.

Therefore, under our experimental conditions (pH>6.5), amelogenin is the crucial factor in controlling the oriented growth of fluoridated hydroxyapatite crystals. Even so, the role of chitosan is likely more than just as an amelogenin carrier. Chitosan in the CS-AMEL system could provide effective protection from enamel erosion because of its pH-responsiveness. The development of caries is associated with a continuous pH change in plaque biofilm due to the accumulation of acid byproducts from metabolism of fermentable carbohydrates. As the pH decreases (in the general range of 5.5-5.0), positive hydrogen ions from the acid bind to the negative phosphate and hydroxyl ions from enamel mineral leading to mineral loss. The potential advantage of having chitosan present on the enamel surface is that the amino group of chitosan could capture the hydrogen ions from the acid, forming a positive protective layer to prevent the diffusion of hydrogen ions to the mineral surface, as well as interacting with amelogenin to avoiding amelogenin loss into the saliva. When normal pH is restored (to the range of 6.3-7.0) by the saliva, the weakly-interacting amelogenin would be released from the chitosan to regulate the remineralization of enamel.

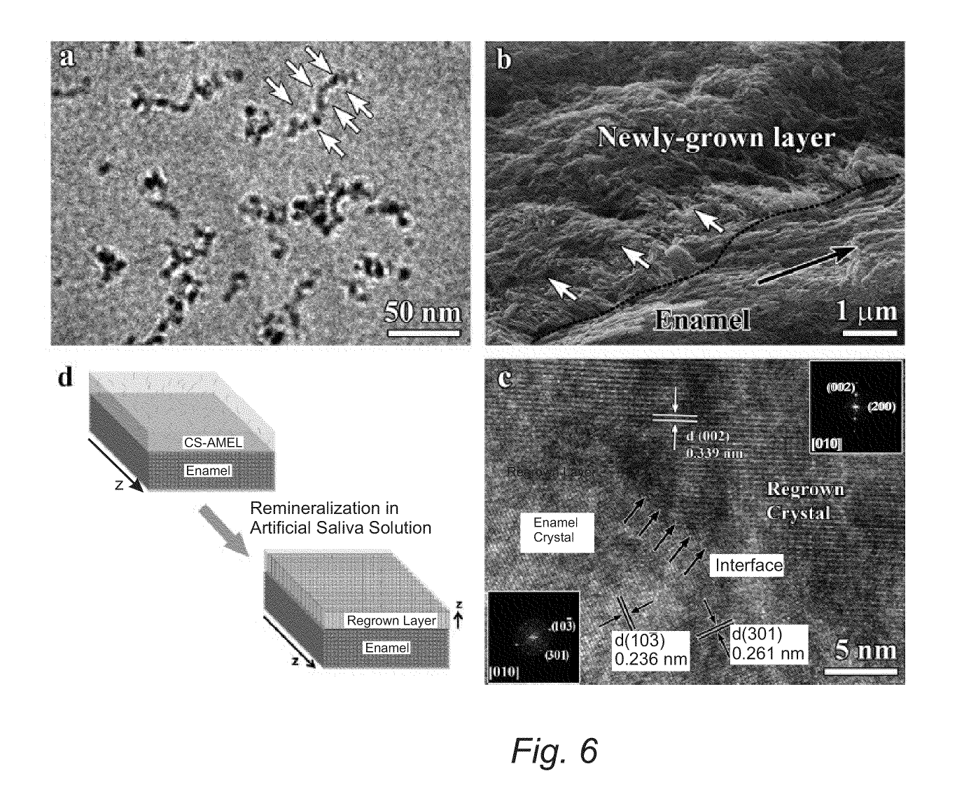

Our HRTEM and SEM analyses provide further insight into the function of amelogenin in remineralization of newly-grown mineral. In the original CS-AMEL hydrogel, we observed linear chains of ˜10 nm nanoclusters, as shown in FIG. 6a. The calcium phosphate (Ca—P) clusters formed in the Ca—P supersaturated condition are thought to be building blocks of both amorphous calcium phosphate as well as apatitic mineral phase. Generally, without a stabilizing agent, these Ca—P clusters aggregate randomly to form plate-like mineral particles. Indeed, we could not find any oriented aggregation of Ca—P clusters in the original chitosan hydrogel in the absence of amelogenin. We suggest that the presence of amelogenin provides an opportunity for stabilization of the precritical clusters at a minimum free energy since the coassembly of Amel-Ca—P clusters imparts kinetic and thermodynamic stability to the system. As a result and as in previous studies, we suggest that amelogenin assemblies stabilized the Ca—P clusters in the CS-AMEL hydrogel and guided their arrangement of clusters into linear chains that eventually evolved into enamel-like co-aligned crystals anchored to the natural enamel substrate.

### 3.4. Continuous Growth of Newly-Formed Crystals on the Enamel

SEM images of the side view of a layer grown in CS-AMEL hydrogel for 3 days (FIG. 6b) indicate that the newly-grown crystals are mostly oriented perpendicular to the surface of the substrate and in non-prismatic orientations (white arrows in FIG. 6b). The interface, indicated by the dotted line, between the repaired layer and the enamel substrate reveals no apparent gap. To further explore the interface microstructure, we used a focused ion beam (FIB) technique to prepare TEM samples for higher resolution analysis. FIG. 6c depicts the HRTEM image of the interface where the new crystals nucleate, clearly exhibiting lattice fringes from the (301) and (10 3̅) planes of the enamel crystal (d=0.261 nm and d=0.236 nm), as well as the (002) plane of the synthetic crystal (d=0.339). The corresponding fast Fourier transform (FFT) images (inset in FIG. 6c) show two different patterns, indicating that the crystals in the enamel and in the fused repaired layer grew with different orientation, which is consistent with the SEM observations in FIG. 6b. Remarkably, the enamel and the newly-grown crystals fused together to form a seamless interface (black arrows in FIG. 6c).

Although the exact growth mechanism remains unresolved, it is clear that amelogenin-stabilized clusters with orientated aggregation are crucial to the continuous growth of new crystals on the enamel. Recent research has shown that calcium-based biominerals can be formed at a templating surface via stable pre-nucleation clusters, with aggregation into an amorphous precursor phase and transformation of this phase into a crystal. Similar to these cluster-growth models, the newly-grown crystals formed in CS-AMEL hydrogel in our experiments started with the aggregation of prenucleation clusters leading to the nucleation of ACP and then the development of oriented apatite crystals. The possible repair processes are schematically presented in FIG. 6d. Initially, the pre-nucleation Ca—P clusters, stabilized by amelogenin, aggregate to form linear chains in the CS-AMEL hydrogel. Subsequently, parts of the cluster aggregates in contact with the enamel surface start to become dense by adopting a closer packing of the clusters. The continuation of this process leads to the formation of an amorphous precursor phase that further fuses with enamel crystals and ultimately transforms into crystalline apatite, which is oriented along the c-axis as directed by amelogenin. As a result, the newly-formed crystals are continuously grown on the enamel crystals and oriented by amelogenin so that their long axes run perpendicular to the enamel surface like natural enamel prisms.

### 3.5. Bonding Strength Between Newly-Grown Layer and Enamel Surface

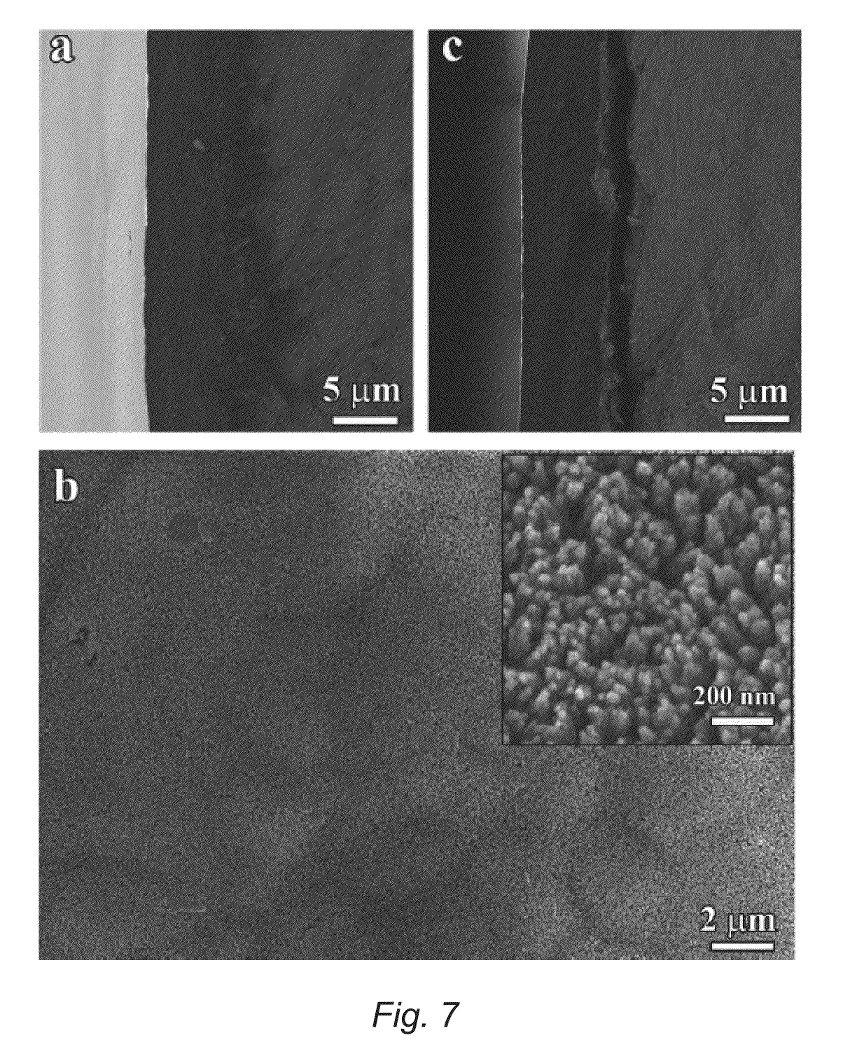

The dense interface between synthetic and natural enamel crystals promoted strong bonding between the newly-grown layer and the tooth surface. FIG. 7 shows the backscattered electron and secondary electron images of the newly-grown layer after ultrasonic treatment (42 kHz, 100 W) for 10 min. The results revealed that the newly-grown layer formed in the CS-AMEL hydrogel was tightly bound to the enamel surface (FIG. 7a), and the organized structure was unaffected by the ultrasonic treatment (FIG. 7b). In contrast, following the same treatment we observed a large gap between the enamel and the repaired layer formed in the chitosan hydrogel without amelogenin (FIG. 7c). In clinical dentistry bonding strength is one of the most important attributes for enamel restorative materials. Due to poor adhesion that leads to gaps at the enamel-restoration interface the currently available materials often have limitation in their durability. These gaps increase the possibility for bacterial leakage and secondary caries, which are the main causes of restoration failure. In the present study, the robust attachment of the newly-grown layer formed in the CS-AMEL hydrogel can potentially improve the durability of restorations and avoid the formation of new caries at the margin of the restoration.

### 3.6. Mechanical Properties of Reconstructed Enamel-Like Layer Repaired by CS-AMEL Hydrogel

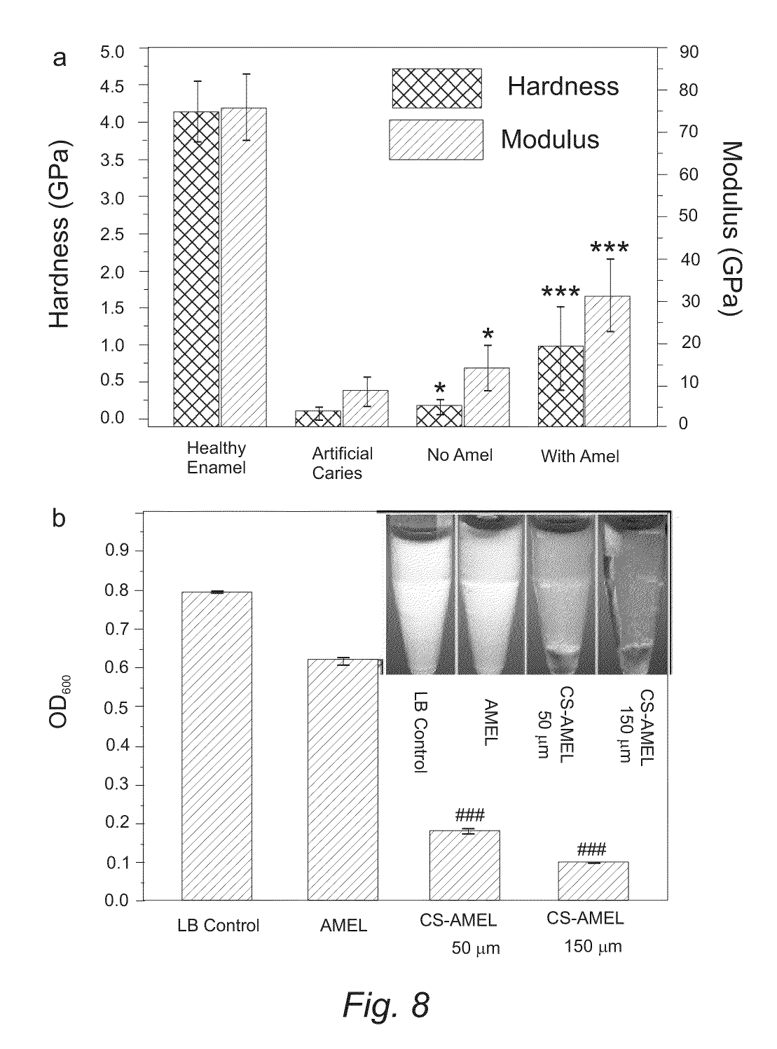

FIG. 8a shows the hardness and elastic modulus of healthy enamel, etched enamel, and the reconstructed enamel-like layer repaired by chitosan hydrogel with and without amelogenin. The hardness and modulus of caries-free enamel slice were estimated to be 4.0 GPa and 70 GPa respectively, and both the hardness and modulus were severely compromised by acid etching (nearly 88% decrease in modulus and 98% decrease in hardness). After mineralization in chitosan hydrogel without amelogenin, we only observed slight increase in the hardness and modulus of etched enamel surface (FIG. 8a). Clearly, the porous structure (FIG. 2c and d) caused by conventional remineralization could not provide a satisfied mechanical function. However, after treatment with amelogenin-chitosan hydrogel for 7 days, the hardness and modulus of the etched enamel surface increased significantly (p<0.001). The modulus increased by nearly 4 times and the hardness was increased by nearly 9 times (FIG. 8a). Although the mechanical properties were not the same as those of native enamel, the repaired enamel treated with CS-AMEL hydrogel showed superior properties compared to the control (without amelogenin) due to the well-organized crystal orientation. The amelogenin and chitosan residues in the repaired layer may limit its mechanical performance, which could potentially be improved by removal of the organic material with proteolytic enzymes. Moreover, in clinical practice, the mechanical properties of repaired layer would be further improved by repetitive application of CS-AMEL hydrogel in order to achieve thicker repaired layer. Further work is needed in order to assess the stability of CS-AMEL hydrogel in the oral cavity.

### 3.7. Antimicrobial Properties of CS-AMEL Hydrogel

Human saliva used as a source for bacteria was cultured in LB medium to examine the antimicrobial properties of CS-AMEL by observing the optical density (OD) values and turbidity (FIG. 8b). After overnight culture, the medium without chitosan gel was opaque due to presence of bacteria, while the medium with chitosan gel was clear (insert in FIG. 8b). The OD value was significantly reduced when the CS-AMEL hydrogel was added to the LB medium (p<0.001, FIG. 8b). These results demonstrate that the CS-AMEL hydrogel can effectively inhibit bacteria growth. We believed that the antimicrobial effect of CS-AMEL hydrogel was attributed to the chitosan. Chitosan has been observed to have antimicrobial activity against a wide variety of bacteria, including streptococci and lactobacilli, which are known as the principal etiological factors of dental caries. Moreover, chitosan has several advantages over other types of antiseptic agents, including a higher antibacterial activity, a broader spectra of activity, a higher killing rate, and lower toxicity toward mammalian cells. Therefore, we expect that the clinical application of the CS-AMEL hydrogel can not only fulfill the superficial enamel reconstruction, but also effectively suppress the bacterial infection and subsequent demineralization.

### 4. Conclusions

In summary, taking advantage of the potential of amelogenin to control organized growth of apatite crystals and the potential antimicrobial activity of chitosan, we have developed a new amelogenin-containing chitosan hydrogel for superficial enamel reconstruction. Amelogenin assemblies stabilized Ca—P clusters in CS-AMEL hydrogel and guided their arrangement into linear chains. These amelogenin Ca—P composite chains further fused with enamel crystal and eventually evolved into enamel-like co-aligned crystals, anchored to the natural enamel substrate through a cluster growth process. The continuous growth of crystals formed an excellent bond between the newly-grown layer and the enamel. Furthermore, the hardness and elastic modulus of etched enamel were increased by 9 and 4 times after treatment with amelogenin-chitosan hydrogel. We anticipate that chitosan hydrogel will provide effective protection against secondary caries because of its pH-responsive and antimicrobial properties. Our studies introduce a promising amelogenin-chitosan hydrogel method for superficial enamel repair and demonstrate the potential of applying protein-directed assembly to the biomimetic reconstruction of complex biomaterials.

### Experimental Showing the Effects of Viscosity and Supersaturation Degree

In the present study, in order to further optimize the CS-AMEL hydrogel to meet the necessary conditions for clinical applicability (i.e. hydrogel stability, shorter preparation and crystallization times), we investigated the effects of viscosity and supersaturation degree in CS-AMEL hydrogel on apatite crystal growth on an acid-etched enamel surface. The size, orientation and composition of newly-grown crystals were studied by scanning electron microscopy (SEM) and X-ray diffraction (XRD). Our long-term objective is to develop strategies for the application of CS-AMEL hydrogel in reconstructing human tooth enamel.

### Materials and Methods

Recombinant full-length porcine amelogenin rP172 was expressed and purified as described previously. Human third molars free of cavities were collected following an Institutional Review Board protocol and acid-etched tooth slices were prepared as described previously. Amelogenin-chitosan hydrogel was prepared by mixing chitosan (1, 2 and 3% m/v), Ca²⁺ (2.5, 5.0, 7.5 and 10 mM) and PO₄ (1.5, 3.0, 4.5 and 6 mM) solutions with amelogenin rP172 (200 μg/ml), followed by stirring at room temperature overnight, and the pH value was adjusted to 6.5 with 1M NaOH. The initial molar ratio of Ca²⁺ to PO₄ ions was kept steady at 1.67; for simplicity we only report the Ca²⁺ concentration in the text that follows. The relative supersaturation degree with respect to hydroxyapatite σ(HAp) was calculated using previously described methods. Twenty μl of chitosan-based hydrogel were carefully applied onto the enamel surface and dried for 15 min in air at room temperature. This is a shorter time than in our previous protocol. The tooth slices were then immersed in 30 ml of artificial saliva solution with 1 mg/L of NaF at 37° C. After 7 days, the tooth slices were removed from the solution, rinsed with running deionized water for 50 s and air-dried.

The morphologies of newly grown crystals were observed by scanning electron microscopy (SEM), which was performed on a field emission scanning electron microscope (JEOL JSM-7001F), operating at an accelerating voltage of 15 kV. The diameter of crystals in the digital SEM images was measured using Photoshop (n>45) and expressed as mean±standard deviation. Student's t-test was applied to identify differences in the sizes of crystals formed in different CS-AMEL hydrogels. The differences were considered statistically significant at p≦0.05 and highly significant at p<0.001. All the statistical analyses were carried out using Origin 8.0 (Origin Lab, Northampton, Mass.) and Microsoft Office Excel 2007.

The composition and orientation of the newly grown crystals were studied by X-ray diffraction (XRD). XRD patterns were recorded on a Rigaku Diffractometer with Cu Kr radiation (λ=1.542 Å) operating at 70 kV and 50 mA with a step size of 0.02°, at a scanning rate of 0.1° s⁻¹ in a 2θ range from 10° to 60°. To estimate the orientation degree of the apatite crystals (JCPDS 09-0432), the intensity (I) of diffraction peaks at 20=25.8° for (002) and 31.8° for (211) were used in a quantitative evaluation. The (002)-(211) intensity ratios in the XRD patterns ware calculated after fitting the peak profiles in a MDI Jade 6.5.

The viscosity of CS-AMEL hydrogel was measured using a dynamic rheometer (TA Instrument, AR2000ex). The settings for viscosity measurement were as follows: parallel plate apparatus=50 mm, gap size=1 mm, temperature=37° C., strain=1%, frequency sweep range=0.1 to 10 Hz. Rheological parameter of storage modulus (G′) was determined and the average storage modulus ( G̅′) was calculated in order to provide a better comparison.

### Results

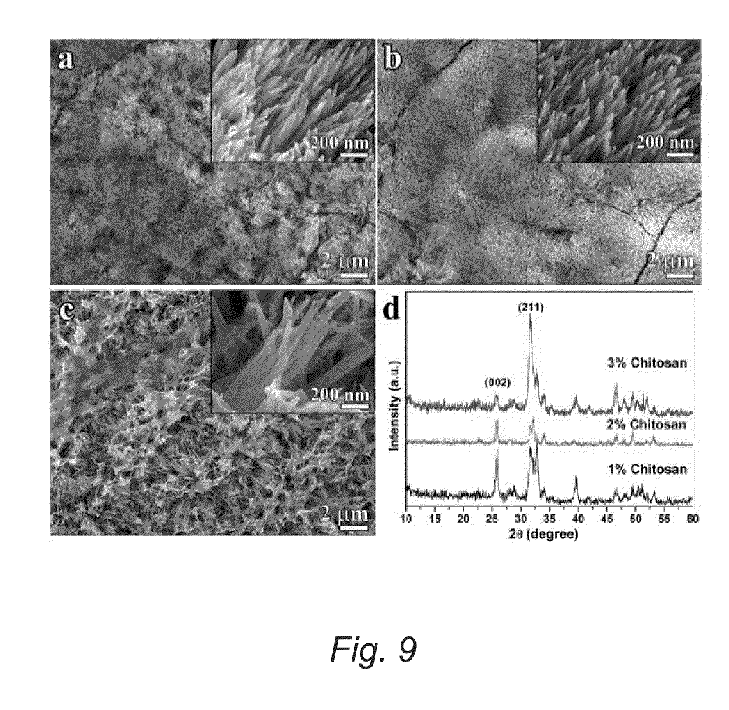

The effect of viscosity on crystal growth was studied in the presence of 1%, 2% and 3% (m/v) chitosan in CS-AMEL hydrogel containing a constant level of [Ca²⁺]=2.5 mM, and 200 μg/ml amelogenin. With increasing chitosan concentration, the average storage modulus of CS-AMEL hydrogel raised from 9.92 to 227.59 Pa, indicating a dramatic increase in viscosity (Table 1). For all the etched tooth samples, after treatment with the different CS-AMEL hydrogels, a synthetic layer composed of hydroxyapatite crystals with diameters of ˜50 nm was observed on the enamel surface (FIG. 9 and Table 1). The majority of the crystals grown in CS-AMEL hydrogel with 1% chitosan had a needle-like shape and were arranged roughly parallel to each other (FIG. 9a). At 2% chitosan, a denser layer composed of more organized crystals was formed on the etched enamel surface (FIG. 9b). When the chitosan concentration increased to 3%, the newly grown layer became a loose aggregate of crystals with a porous structure (FIG. 9c). SEM observations showed an obvious change in the crystal orientation, which was further confirmed by XRD analyses. The (002)-(211) intensity ratios of the synthetic crystals formed in different CS-AMEL hydrogels increased from 0.9 to 1.71 and then dropped to 0.17 when the chitosan concentration was increased from 1% to 2% and then 3%, respectively (Table 1). Based on the SEM and XRD results, it can be seen that the orientation of the newly grown crystals was affected by the hydrogel viscosity. The best crystal orientation was achieved in the sample remineralized in CS-AMEL hydrogel with 2% chitosan.

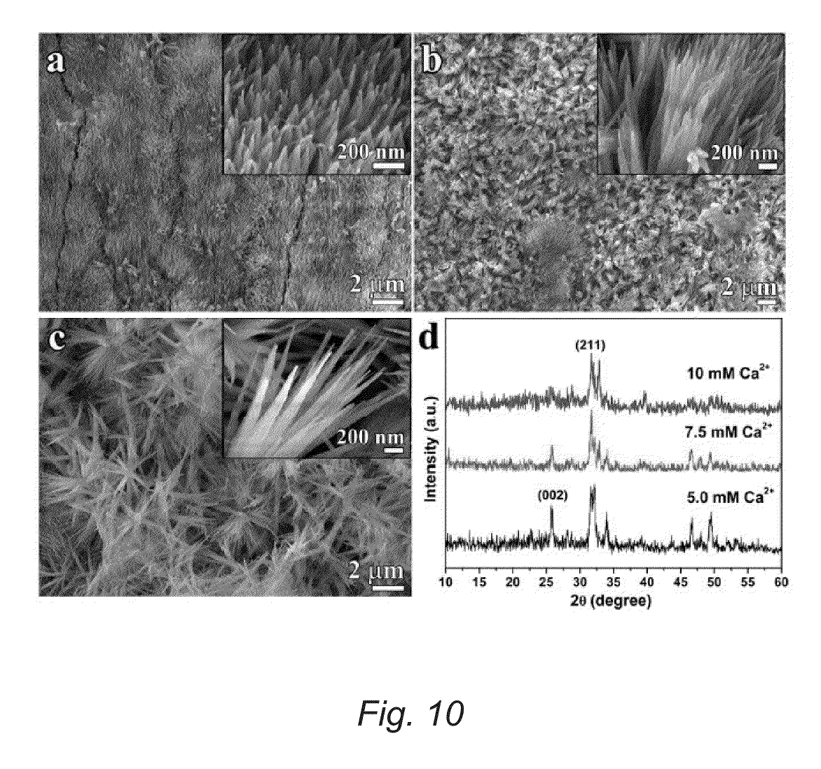

With the optimal chitosan concentration determined to be 2% m/v, with an average storage modulus of 17.55 (Pa) the effects of supersaturation degree on the crystal morphology and organization were assayed for [Ca²⁺] values ranging from 2.5 mM to 10.0 mM in the CS-AMEL hydrogel with 2% (m/v) of chitosan and 200 μg/ml of amelogenin. The increase in supersaturation degree resulted in a significant increase in crystal diameter from 52.77±10.78 nm to 95.03±26.30 nm (Table 2), and also dramatically disrupted the organization of crystals formed in the hydrogel. At low supersaturation (σ(HAp)<10.06, [Ca²⁺]<5.0 mM), organized apatite crystals with enamel-like structure formed on the etched enamel surface (FIGS. 9b and 10a). At a σ(HAp) of 11.07 ([Ca²⁺]=7.5 mM), the newly grown layer was loose despite the appearance of some enamel-like crystal bundles. Increasing the σ(HAp) to 11.76 ([Ca²⁺]=10.0 mM) resulted in a porous coating with irregularly packed crystals. From the XRD results, the (002)-(211) intensity ratios dropped from 1.71 to 0.45 with increasing σ(HAp) from 8.23 to 11.07, revealing a significant reduction in the crystal orientation degree. There was no detectable (002) peak observed in the XRD pattern of the newly grown layer formed in CS-AMEL hydrogel with σ(HAp) of 11.76 ([Ca²⁺]=10.0 mM). The SEM and XRD results indicated σ(HAp) at 10.06 ([Ca²⁺]=5.0 mM) was the upper limit for preserving the enamel-like oriented crystal structure.

### Discussion

Amelogenin-chitosan (CS-AMEL) hydrogel has been reported as a promising material for superficial enamel repair. During enamel remineralization in CS-AMEL hydrogel, the amelogenin assemblies promote the formation of an organized enamel-like microstructure which is crucial for successful enamel reconstruction.

Due to the need to keep the amelogenin on the enamel surface for effective repair, the stability of the hydrogel carrier is a necessary prerequisite for clinical application. Since chitosan is the major component of the CS-AMEL hydrogel, one way to improve stability is to raise the viscosity of the hydrogel by increasing the chitosan concentration. In our previous study, the CS-AMEL hydrogel with low chitosan concentration (1% m/v) needed a relatively long time (2 hours) to be stabilized on the enamel surface. This highly flexible hydrogel ( G̅′=9.92 Pa) had difficulty stabilizing on the etched enamel surface in a short time (15 min), leading to the loss of a small amount of amelogenin. For this reason, the new layer grown after 15 min in the CS-AMEL hydrogel with 1% chitosan did not present the best crystal orientation. Increasing the chitosan concentration to 2% (m/v) improved the stability of the CS-AMEL hydrogel increasing its viscosity ( G̅′=17.55 Pa). During the enamel remineralization in the artificial saliva solution, we observed a transparent layer of CS-AMEL hydrogel covering the enamel surface. As a result, with this concentration of chitosan (2%), the amelogenin assemblies were protected inside the hydrogel and were able to produce a dense layer composed of organized crystals. When the chitosan concentration increased to 3%, the viscosity of CS-AMEL hydrogel was increased dramatically ( G̅′=227.59 Pa), however a relatively loose structure was observed. It has been reported that chitosan is able to form hydrophobic aggregation at a high concentration. This association between macromolecular chains appears to have decreased the mobility of the chains towards others and hence sterically hindered the oriented growth of apatite crystals.

The optimal size and orientation of the synthetic nanocrystals will provide a newly formed repaired layer with better mechanical properties. Therefore, it is important to study the factors that affect the size and orientation of crystals grown in CS-AMEL matrix. Recent research has shown that the nanocrystal structures were sensitive to the degree of supersaturation and that dense nanorod crystals only formed in the supersaturation degree range from 8-12. In a CS-AMEL system, the region of supersaturation degree for forming the oriented crystals is narrower. A supersaturation degree higher than 10.06 ([Ca²⁺]>5 mM) will result in random crystals with a larger size and irregular structure due to the disruption of the balance between the Ca—P prenucleation cluster and its stabilizer, amelogenin. Unlike the classical nucleation process, the formation of enamel-like apatite crystals in a CS-AMEL matrix proceeds through a continuous growth process that involves the amelogenin-mediated formation, aggregation and transformation of Ca—P prenucleation clusters. It is noteworthy that the regulatory effects of amelogenin on the growth of calcium phosphate crystals are dose-dependent. A higher supersaturation degree will thermodynamically accelerate the crystal nucleation, resulting in numerous excessive Ca—P clusters, which cannot be stabilized by the limited amount of protein, leading to the random growth of crystals with a larger size and irregular structure. As a result, in our experiment, it was difficult to find an organized enamel-like microstructure when the [Ca²⁺] was higher than 5 mM.

### CONCLUSION

In a CS-AMEL system, enamel-like organized apatite crystals can only be formed under certain conditions, which are determined by the synergetic effects of hydrogel viscosity and supersaturation degree with respect to apatite. A suitable concentration of chitosan will provide the CS-AMEL hydrogel with optimal stability, ensuring the amelogenin-mediated growth of oriented crystals. On the other hand, a higher degree of supersaturation will thermodynamically promote the formation of random crystals with a larger size and irregular structure. The optimal conditions to produce organized enamel-like crystals in a CS-AMEL hydrogel are: 2% (w/v) chitosan, 2.5 mM calcium, and 1.5 mM phosphate (supersaturation degree=8.23), with 200 μg/ml of full-length amelogenin.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A composition for treating dental caries, early dental carious and erosive lesions, and enamel defects resulting from genetic diseases, the composition comprising:
an amelogenin;
a chitosan;
water; and
a sufficient amount of a pH adjusting component such that the composition has a pH greater than about 6.0, the composition forming an organized mineral layer when contacting an enamel surface.

2. The composition of claim 1 wherein the amelogenin is present in an amount from about 0.03 percent to about 0.4 percent of the total weight of the composition and the chitosan is present in an amount from about 0.2 to about 3 percent of the total weight of the composition.

3. The composition of claim 2 further comprising:
a calcium-containing compound that provides calcium ion when dissolved in water; and
a phosphate containing compound that provides phosphate ions when dissolved in water.

4. The composition of claim 3 wherein the calcium containing compound is present in an amount from about 0.01 percent to about 0.2 percent of the total weight of the composition and the phosphate containing compound is present in an amount from about 0.01 percent to about 0.2 percent of the total weight of the composition.

5. The composition of claim 1 wherein the amelogenin is a recombinant full-length amelogenin, and isoforms thereof.

6. The composition of claim 1 wherein the amelogenin is a truncated amelogenin or an alternatively spliced isoform.

7. The composition of claim 1 wherein the amelogenin is a leucine rich amelogenin polypeptide (LRAP).

8. The composition of claim 1 wherein the chitosan has an average molecular weight (Mv) from about 150,000 to 400,000 Daltons.

9. The composition of claim 1 wherein the composition is a hydrogel.

10. The composition of claim 1 further comprising additional protein components.

11. The composition of claim 1 wherein the additional protein components are selected from the group consisting of enamelin, ameloblastin, enamel proteases, chitosanolytic enzymes, and combinations thereof.

12. The composition of claim 11 wherein the additional protein components are individually or collectively present in an amount from about 0.005 percent to about 0.1 percent of the total weight of the composition.

13. The composition of claim 1 wherein the chitosan has a formula described by a partially acetylated polysaccharide having formula I: 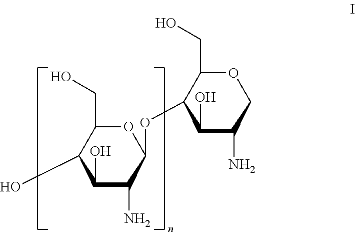
wherein n is from about 500 to 2500.

14. The composition of claim 13 wherein the partially acetylated polysaccharide having formula I has a degree of acetylation less than about 35 percent.

15. The composition of claim 13 wherein the partially acetylated polysaccharide having formula I has a degree of acetylation from about 5 to 35 percent.

16. A method comprising:
a) contacting an enamel coated substrate with a composition including:
an amelogenin;
a chitosan;
water;
a sufficient amount of a pH adjusting component such that the composition has a pH greater than about 6.0; and
b) allowing the composition to air dry.

17. The method of claim 16 further comprising:
a calcium-containing compound that provides calcium ion when dissolved in water; and
a phosphate containing compound that provides phosphate ions when dissolved in water.

18. The method of claim 16 further comprising contacting the substrate with a base for a first period of time.

19. The method of claim 18 further comprising contacting the substrate with a component selected from the group consisting of calcium-containing compound, a phosphate containing compound, and combinations thereof for a second period of time.

20. The method of claim 19 further comprising contacting the substrate with saliva or a solution including water and a component selected from the group consisting of electrolytes, mucus, glycoproteins, enzymes, antibacterial compounds, and combinations thereof.

21. The method of claim 16 wherein the substrate is a tooth.


SEQUENCE LISTING (8 sequences)

SEQ ID NO: 1 - protein, 189 aa, Sus scrofa
MGTWILFACLLGAAFSMPLPPHPGHPGYINFSYEVLTPLKWYQNMIRHPYTSYGYEPMGGWLHHQIIPVVSQQTPQSHALQPHHHIPMVPAQQPGIPQQPMMPLPGQHSMTPTQHHQPNLPLPAQQPFQPQPVQPQPHQPLQPQSPMHPIQPLLPQPPLPPMFSMQSLLPDLPLEAWPATDKTKREEVD

SEQ ID NO: 2 - protein, 184 aa, Bos taurus
FACLLGAAYSMPLPPHPGHPGYINFSYEVLTPLKWYQNMLRYPYPSYGYEPVGGWLHHQIIPVVSQQSPQNHALQPHHHNPMVPAQQPVVPQQPMMPVPGQHSMTPIQHHQPNLPLPAQQSFQPQPIQPQPHQPLQPQPPVHPIQRLPPQPPLPPIFPMQPLPPVLPDLPLEAWPATDKTKREE

SEQ ID NO: 3 - protein, 191 aa, Homo sapiens
MGTWILFACLLGAAFAMPLPPHPGHPGYINFSYEVLTPLKWYQSIRPPYPSYGYEPMGGWLHHQIIPVLSQQHPPTHTLQPHHHIPVVPAQQPVIPQQPMMPVPGQHSMTPIQHHQPNLPPPAQQPYQPQPVQPQPHQPMQPQPPVHPMQPLPPQPPLPPMFPMQPLPPMLPDLTLEAWPSTDKTKREEVD

SEQ ID NO: 4 - protein, 198 aa, Pongo pygmaeus
ACLLGAAFAMPLPPHPGHPGYINFSYENSHSQAINVDRTALVLTPLKWYQSIRPPYPSYGYEPMGGWLHHQIIPVLSQQHPPTHTLQPHHHIPVVPAQQPVIPQQPMMPVPGQHSMTPTQHHQPNLLPPAQQPYQPQPVQPQPHQPMQPQPPVHPMQPLPPQPPLPPMFPMQPLPPMLPDLTLEAWPSTDKTKREEVD

SEQ ID NO: 5 - protein, 205 aa, Pan troglodytes
MGTWILFACLLGAAFAMPLPPHPGHPGYINFSYENSHSQAINVDRTALVLTPLKWYQSIRPPYPSYGYEPMGGWLHHQIIPVLSQQHPPTHTLQPHHHIPVVPAQQPVIPQQPMMPVPGQHSMTPIQHHQPNLPPPAQQPYQPQPVQPQPHQPMQPQPPVHPMQPLPPQPPLPPMFPMQPLPPMLPDLTLEAWPSTDKTKREEVD

SEQ ID NO: 6 - protein, 195 aa, Rattus norvegicus
MGTWILFACLLGAAFAMPLPPHPGSPGYINLSYEVLTPLKWYQSMIRQPHPPSHTLQPHHHLPVVPAQQPVAPQQPMMPVPGHHSMTPTQHHQPNIPPSAQQPFQQPFQPQAIPPQSHQPMQPQSPLHPMQPLAPQPPLPPLFSMQPLSPILPELPLEAWPATDKTKREEVAFSPMKWYQGTARHPLNMETTTEK

SEQ ID NO: 7 - protein, 210 aa, Mus musculus
MGTWILFACLLGAAFAMPLPPHPGSPGYINLSYEKSHSQAINTDRTALVLTPLKWYQSMIRQPYPSYGYEPMGGWLHHQIIPVLSQQHPPSHTLQPHHHLPVVPAQQPVAPQQPMMPVPGHHSMTPTQHHQPNIPPSAQQPFQQPFQPQAIPPQSHQPMQPQSPLHPMQPLAPQPPLPPLFSMQPLSPILPELPLEAWPATDKTKREEVD

SEQ ID NO: 8 - protein, 56 aa, Sus scrofa
MPLPPHPGHPGYINFSYEVLTPLKWYQNMIRHPSLLPDLPLEAWPATDKTKREEVD